(19) United States
(12) Patent Application Publication
(10) Pub. No.: US 2024/0253296 A1
(43) Pub. Date: Aug. 1, 2024

(54) BIO-INK PRINTING

(71) Applicants: HEWLETT-PACKARD DEVELOPMENT COMPANY, L.P., Spring, TX (US); NANYANG TECHNOLOGICAL UNIVERSITY, Singapore (SG)

(72) Inventors: Wei Long NG, Singapore (SG); Xi HUANG, Singapore (SG); Viktor SHKOLNIKOV, Palo Alto, CA (US); Roberto A. PUGLIESE, Corvallis, OR (US); Mackenzie JOHNSON, Corvallis, OR (US); Wai Yee YEONG, Singapore (SG)

(73) Assignees: HEWLETT-PACKARD DEVELOPMENT COMPANY, L.P., Spring, TX (US); NANYANG TECHNOLOGICAL UNIVERSITY, Singapore (SG)

(21) Appl. No.: 18/559,987

(22) PCT Filed: May 11, 2021
(86) PCT No.: PCT/US2021/031671

§ 371 Date: Nov. 9, 2023

(51) Int. Cl. B29C 64/112; B29C 64/209; B29C 64/245; B29C 64/255; B29K 101/00; B33Y 10/00; B33Y 30/00; B33Y 70/10; C12N 5/00

(52) U.S. Cl. CPC B29C 64/112; B29C 64/209; B29C 64/245; B29C 64/255; B33Y 10/00; B33Y 30/00; C12N 5/0062; B29K 2101/00; B33Y 70/10; C12N 2513/00; C12N 2533/30

(57) ABSTRACT

The present disclosure is drawn to bio-ink printer components, methods of printing bio-inks, and multi-fluid live cell printing systems. In one example, a bio-ink printer component can include a bio-ink and a bio-ink ejector fluidly connected or connectable to the bio-ink. The bio-ink can include a buffer solution that is suitable for live cells, and a polymer that includes polyvinylpyrrolidone, polyvinyl alcohol, polyvinyl sulfate, polyethylene glycol, polyester, poly(dimethylsiloxane), cellulose, polysaccharide, or a combination thereof. The bio-ink ejector can include an ejection nozzle and a thermal resistor positioned to heat the bio-ink to form a vapor bubble to eject a droplet of bio-ink from the ejection nozzle.

### BACKGROUND

Bio-printing is used for many purposes including performing various types of assay testing, toxicology testing, disease modelling, fundamental biology studies, in-vitro drug screening, and others. The ability to deposit living cells using bio-printers can be very useful in these applications. For example, automated fabrication of in-vitro tissue models using 3D bio-printing of living cells can be very useful for creating tissue models in a scalable and repeatable manner. Demand for bio-printing is expected to increase significantly in the future. The recent prevalence of COVID-19 cases and the increasing prevalence of chronic diseases are some of the many factors that may contribute to this growth in demand for in-vitro testing models.

### BRIEF DESCRIPTION OF THE DRAWING

FIGS. 1A-1D show a schematic cross-sectional view of an example bio-ink printer component in accordance with the present disclosure;

FIG. 2 is a flowchart illustration and example method of printing bio-ink in accordance with the present disclosure; and

FIG. 3 is a schematic cross-sectional view of an example multi-fluid live cell printing system in accordance with the present disclosure.

### DETAILED DESCRIPTION

The present disclosure describes bio-ink printer components and methods of printing bio-ink. The bio-ink printer components can be print cartridges in some examples, while in other examples the bio-ink printer components can include additional elements, and in some examples the bio-ink printer components can include an entire printer. In one example, a bio-ink printer component includes a bio-ink and a bio-ink ejector. The bio-ink includes a buffer solution that is suitable for live cells, and a polymer that includes polyvinylpyrrolidone, polyvinyl alcohol, polyvinyl sulfate, polyethylene glycol, polyester, poly(dimethylsiloxane), cellulose, polysaccharide, or a combination thereof. The bio-ink ejector is fluidly connected or connectable to the bio-ink. The bio-ink ejector includes an ejection nozzle and a thermal resistor positioned to heat the bio-ink to form a vapor bubble to eject a droplet of bio-ink from the ejection nozzle. In certain examples, the bio-ink can also include live cells. In some examples, the polymer can be present in an amount from about 1 wt % to about 3 wt % with respect to the total weight of the bio-ink. The buffer solution can include phosphate buffered saline (PBS), citric acid buffer, Tris buffer, HEPES buffer, CABS buffer, CAPS buffer, AMP buffer, CAPSO buffer, CHES buffer, AMPSO buffer, TABS buffer, AMPD buffer, TAPS buffer, HEPBS buffer, Bicine buffer, Gly-Gly buffer, Tricine buffer, EPPS buffer, TEA buffer, POPSO buffer, HEPPSO buffer, TAPSO buffer, MOBS buffer, DIPSO buffer, HEPES buffer, TES buffer, MOPS buffer, BES buffer, Bis-Tris Propane buffer, MOPSO buffer, PIPES buffer, ACES buffer, ADA buffer, Bis-Tris buffer, MES buffer, or a combination thereof in some examples. In further examples, the bio-ink printer component can also include a substrate positioned to receive the droplet of the bio-ink ejected from the ejection nozzle. The substrate can include a well-plate, a petri dish, a layer of previously-printed cells, glass, hydrogel, or a cellular scaffold. The substrate can be positioned or positionable at a distance from about 5 mm to about 20 mm away from the ejection nozzle. In a particular example, the substrate is positioned at a distance from about 5 mm to about 20 mm away from the ejection nozzle. In other examples, the ejection nozzle can have a width from about 20 micrometers to about 80 micrometers.

The present disclosure also describes methods of printing bio-ink. In one example, a method of printing bio-ink includes ejecting a droplet of a bio-ink from a bio-ink ejector. The bio-ink ejector includes an ejection nozzle and a thermal resistor positioned to heat the bio-ink to form a vapor bubble to eject the droplet of bio-ink from the ejection nozzle. The bio-ink includes live cells, a buffer solution that is suitable for live cells, and a polymer that includes polyvinylpyrrolidone, polyvinyl alcohol, polyvinyl sulfate, polyethylene glycol, polyester, poly(dimethylsiloxane), cellulose, polysaccharide, or a combination thereof. In certain examples, the live cells can be present in the bio-ink at a concentration from about 1 million cells/mL to about 4 million cells/mL, and the polymer can be present in an amount from about 1 wt % to about 3 wt % with respect to the total weight of the bio-ink. In further examples, the buffer solution can include phosphate buffered saline (PBS), citric acid buffer, Tris buffer, HEPES buffer, CABS buffer, CAPS buffer, AMP buffer, CAPSO buffer, CHES buffer, AMPSO buffer. TABS buffer, AMPD buffer, TAPS buffer, HEPBS buffer, Bicine buffer, Gly-Gly buffer, Tricine buffer, EPPS buffer, TEA buffer, POPSO buffer, HEPPSO buffer, TAPSO buffer, MOBS buffer, DIPSO buffer, HEPES buffer, TES buffer, MOPS buffer, BES buffer, Bis-Tris Propane buffer, MOPSO buffer, PIPES buffer, ACES buffer, ADA buffer, Bis-Tris buffer, MES buffer, or a combination thereof. In certain examples, the droplet can form a filament stretching to a distance from about 2 mm to about 6 mm from the ejection nozzle before the filament separates from the ejection nozzle. In some examples, the droplet can be ejected onto a substrate and the live cells can have a viability from about 90% to about 100% after being ejected onto the substrate.

The present disclosure also discloses multi-fluid live cell printing systems. In one example, a multi-fluid live cell printing system includes a first bio-ink reservoir containing a first bio-ink. The first bio-ink includes a first type of live cells, a buffer solution that is suitable for live cells, and a polymer that includes polyvinylpyrrolidone, polyvinyl alcohol, polyvinyl sulfate, polyethylene glycol, polyester, poly(dimethylsiloxane), cellulose, polysaccharide, or a combination thereof. The system also includes a second bio-ink reservoir containing a second bio-ink. The second bio-ink includes a buffer solution that is also suitable for live cells. The system also includes a first bio-ink ejector to eject the first bio-ink from the first bio-ink reservoir. The first bio-ink ejector includes a first ejection nozzle and a first thermal resistor positioned to heat the first bio-ink to form a vapor bubble to eject a droplet of the first bio-ink from the first ejection nozzle. The system also includes a second bio-ink ejector to eject the second bio-ink from the second bio-ink reservoir. In certain examples, the polymer can be present in the first bio-ink at an amount from about 1 wt % to about 3 wt % with respect to the total weight of the first bio-ink. In further examples, the second bio-ink can include a second type of live cells, a polymer including polyvinylpyrrolidone, polyvinyl alcohol, polyvinyl sulfate, polyethylene glycol, polyester, poly(dimethylsiloxane), cellulose, polysaccharide, or a combination thereof, and the second bio-ink ejector can include a second ejection nozzle and a second thermal resistor positioned to heat the second bio-ink to form a vapor bubble to eject a droplet of the second bio-ink from the second ejection nozzle.

It is noted that when discussing bio-inks, bio-ink ejectors, bio-ink printer components, methods, and systems, these discussions can be considered applicable to other examples whether or not they are explicitly discussed in the context of that example unless expressly indicated otherwise. Thus, for example, when discussing a bio-ink composition, such disclosure is also relevant to and directly supported in context of bio-ink printer components, systems, methods of printing bio-ink, and vice versa. Furthermore, for simplicity and illustrative purposes, the present disclosure is described by referring mainly to certain examples. In the following description, numerous specific details are set forth in order to provide a thorough understanding of the present disclosure. It will be readily apparent however, that the present disclosure can be practiced without limitation to some of these specific details. In other instances, certain methods, compounds, compositions, and structures have not been described in detail so as not to unnecessarily obscure the present disclosure.

### Bio-Ink Printer Components

As mentioned above, the bio-ink printer components described herein can include portions of a bio-ink printer or an entire bio-ink printer, depending on the particular example. In some examples, the bio-ink printer component can be a print cartridge that includes a bio-ink reservoir and a bio-ink ejector for printing the bio-ink. In other examples, the bio-ink printer component can include a print cartridge and additional elements, such as a print substrate. In further examples, the bio-ink printer component can include an entire printer. The bio-ink printer components described herein can be used to print bio-inks that can include live cells. Such printing of live cells can be useful in a variety of applications, including performing various assays, forming multiplexed cell microarrays, in-vitro drug screening, printing engineered tissues, and others. In some examples, three-dimensional tissues can be formed by printing multiple layers of multiple different types of living cells.

Printing bio-inks with live cells can involve several concerns. One concern is the viability of the cells. In some cases, the cell viability can be affected by the compatibility of the cells with other ingredients in the bio-ink. The cell viability can also be affected by damage to the cells that may occur when the bio-ink is ejected from a printer or by the collision of the bio-ink with the substrate on which the bio-ink is printed. Additional concerns include the ability to precisely and accurately print droplets of the bio-ink. Some ingredients in the bio-ink can also affect printability of the bio-ink, as some ingredients may damage the printer or affect the viscosity, surface tension, or other properties of the bio-ink to make the bio-ink more difficult to print.

The bio-inks described herein can provide good printability from a thermal jetting bio-ink ejector. This type of ejector can include a thermal resistor that can heat the bio-ink to form a small vapor bubble at the thermal resistor. This vapor bubble can displace a small volume of bio-ink, and thereby push a volume of the bio-ink out of a nozzle. The bio-ink ejected from the nozzle can form a droplet that is propelled from the nozzle onto a print substrate. In some examples, the bio-ink ejector can operate similarly to a thermal inkjet printer. The nozzle of the ejector can be quite small, such as from about 20 μm to about 80 μm in diameter. Therefore, the ejector can eject small droplets at a high resolution. The thermal resistor can also be controlled precisely to provide a desired drop volume. The time for forming a vapor bubble and ejecting a drop of bio-ink can be very short, allowing for fast printing of many droplets in succession. The bio-inks have been successfully printed from this type of bio-ink ejector without clogging or other printability issues.

The bio-inks described herein have also been found to provide good cell viability. For example, when a bio-ink including live cells is printed onto a substrate, the portion of cells that are still alive after being printed on the substrate can be from about 90% to 100% of the cells that were alive in the ink before printing.

A particular polymer can be included in the bio-inks described herein to impart several useful properties to the bio-inks. The polymer can have an effect of increasing the effective surface tension of the bio-ink when the bio-ink is under high shear. For example, the bio-ink can be under high shear when a drop of bio-ink is ejected from the bio-ink ejector. The “effective surface tension” can include the effects of both the surface tension of the bio-ink and the dynamic surface elasticity of the bio-ink. The surface tension and the dynamic surface elasticity can both work together to counter the increase in surface area of the bio-ink. Therefore, these forces counter the breaking of the liquid filament and formation of smaller droplets when the bio-ink is printed. These forces also slow the bio-ink down as the filament stretches. Without being bound to a particular mechanism, in some examples the polymer can increase the surface tension and/or dynamic surface elasticity of the bio-ink because the polymer chains can coil, tangle, and move past one another. The molecular weight of the polymer, concentration of the polymer in the bio-ink, and the shear forces applied to the bio-ink during printing can also affect the dynamic surface elasticity. Several examples of polymers that can be used in the bio-ink include polyvinylpyrrolidone, polyvinyl alcohol, polyvinyl sulfate, polyethylene glycol, polyester, poly(dimethylsiloxane), cellulose, polysaccharide, and combinations thereof. Another useful property provided by the polymer is increased viscosity of the bio-ink. This can help to reduce splashing and splattering of bio-ink drops on the print substrate. It has also been found that bio-ink that includes the polymer can form a long stretched-out filament extending from the ejector nozzle when a drop of the bio-ink is ejected. In some examples, this filament of bio-ink can extend from 2 mm to 6 mm away from the nozzle before the filament detaches and becomes a free drop. This formation of a long filament can be useful because the bio-ink drop can be stabilized by staying attached to the nozzle for a longer time, which can help prevent the droplet from being blown off course by air currents. The stretching effect can also slow down the velocity of the drop before the drop hits the print substrate. In some cases, this can increase the viability of cells in the bio-ink drop. In summary, the bio-inks described herein can provide good printability, high cell viability, high accuracy and precision of drop placement, and low splattering of bio-ink drops.

In certain examples, a bio-ink printer component can include a bio-ink and a bio-ink ejector fluidly connected or connectable to the bio-ink. The bio-ink can include a buffer solution that is suitable for live cells and a polymer. As explained above, the polymer can include polyvinylpyrrolidone, polyvinyl alcohol, polyvinyl sulfate, polyethylene glycol, polyester, poly(dimethylsiloxane), cellulose, polysaccharide, or a combination thereof. The bio-ink ejector can include an ejection nozzle and a thermal resistor positioned to heat the bio-ink. The thermal resistor can be used to quickly heat the bio-ink so that a vapor bubble forms and ejects a droplet of bio-ink from the ejection nozzle.

As used herein, “fluidly connected or connectable” can refer to a bio-ink ejector that is either connected to a supply of bio-ink in a way that allows the bio-ink to flow from the supply to the ejector, or which is designed to be connected to a supply of bio-ink in this way. In some cases, the bio-ink may be packaged separately from the bio-ink ejector. However, the bio-ink ejector can be designed to be fluidly connected to the bio-ink at some point so that the bio-ink can be ejected from the bio-ink ejector. In other examples, a bio-ink printer component can include a reservoir of bio-ink that is already attached and connected to the bio-ink ejector.

In some examples, the bio-ink can include live cells. Thus, the live cells can be deposited onto a substrate by printing the bio-ink using the bio-ink ejector. However, in certain examples the bio-ink can be supplied without live cells. This can allow a user to add a desired type of live cells to the bio-ink before printing the bio-ink. For example, a bio-ink can be formulated including a buffer solution that is suitable for live cells and the polymer as described above, but the bio-ink can be devoid of live cells. This bio-ink can be supplied to a user separately from the bio-ink ejector or together with the bio-ink ejector. The user can then select a desired type of live cells and add the live cells to the bio-ink. The bio-ink, now including the live cells, can then be printed using the bio-ink ejector.

The bio-ink can also be free of ingredients that would inhibit printing using an ejector with a thermal resistor as described above. In some cases, cell growth medium can include ingredients that can inhibit printing with this type of ejector. Such ingredients can include amino acids, vitamins, glucose, serum (such as fetal bovine serum) which can include a variety of biological molecules such as proteins, antibodies, and others. As mentioned above, the bio-ink can include a buffer solution that is suitable for live cells. The buffer solution can be any type of buffer solution that does not inhibit printing with an ejector having a thermal resistor. In some examples, the buffer solution can be made up of small molecules, such as molecules having a molecular weight of 900 g/mol or less.

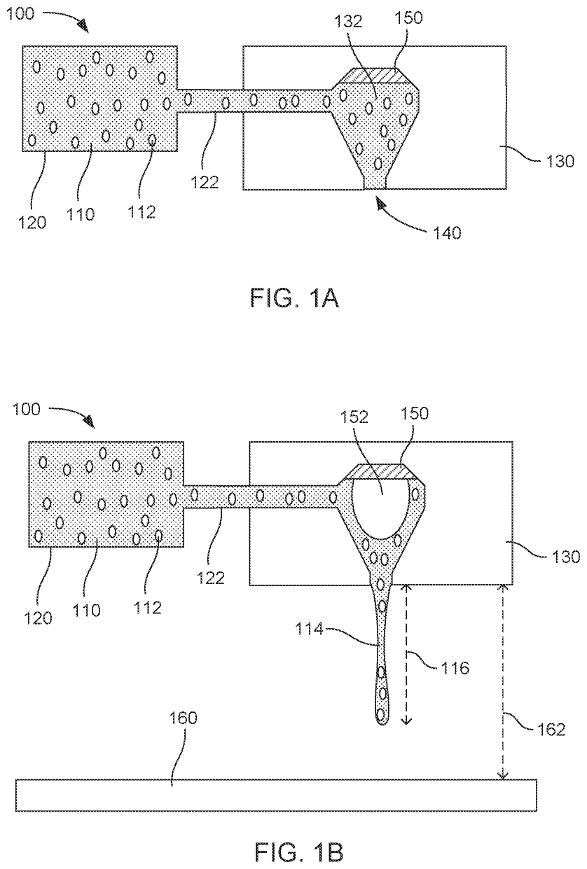

FIG. 1A shows a schematic cross-sectional view of one example bio-ink printer component 100 in accordance with the present disclosure. The bio-ink printer component includes a bio-ink 110 that is contained in a bio-ink reservoir 120. The bio-ink reservoir is fluidly connected to a bio-ink ejector 130 by a bio-ink channel 122. The bio-ink ejector includes an ejection nozzle 140 and a thermal resistor 150. The thermal resistor is positioned to heat bio-ink in an ejection chamber 132 to form a vapor bubble to eject a droplet of bio-ink from the ejection nozzle. In this example, the bio-ink includes live cells 112. As explained above, the bio-ink can also include a buffer solution that is suitable for live cells and a polymer including polyvinylpyrrolidone, polyvinyl alcohol, polyvinyl sulfate, polyethylene glycol, polyester, poly(dimethylsiloxane), cellulose, polysaccharide, or a combination thereof.

FIG. 1B shows the same example bio-ink printer component 100 as the thermal resistor 150 is activated to heat the bio-ink 110. This forms a vapor bubble 152 in the ejection chamber 132. The vapor bubble displaces a volume of the bio-ink and pushes the bio-ink out of the ejection nozzle 140. As mentioned above, the bio-ink can stretch to form a filament 114 that remains connected to the ejection nozzle until the filament stretches to a certain length 116. This figure also shows a substrate 160 positioned at a distance 162 from the bio-ink ejector to receive the bio-ink that is ejected from the ejection nozzle.

FIG. 1C shows the example bio-ink printer component 100 at a moment after the bio-ink 110 ejected from the ejection nozzle 140 has detached, forming a free droplet 118. In practice, the bio-ink may separate into multiple smaller droplets before the droplets impact the substrate 160. Finally, FIG. 1D shows the bio-ink printer component and substrate after the droplet has landed on the substrate. In this figure, the vapor bubble 152 has collapsed and an additional volume of bio-ink is drawn from the bio-ink reservoir 120 into the ejection chamber 132 to replace the volume of the vapor bubble. It is noted that in these figures, the elements of the bio-ink printer component are not drawn to scale. Accordingly, the relative size of elements such as the bio-ink reservoir, thermal resistor, ejection nozzle, live cells, and others can differ from the sizes shown in the figures. Additionally, the figures merely show an example configuration of a bio-ink printer component, and many other configurations and designs can also be used. Bio-ink printer components can also include a variety of additional elements that are not shown in the figures, such as elements similar to those used in thermal inkjet printers.

The bio-ink printer components described herein can be used in a variety of applications, such as multiplexed cell microarrays, in-vitro drug screening, printing engineered tissues, and others. Accordingly, it can be useful to include a variety of types of live cells in the bio-ink. In some examples, the live cells can be primary cells derived directly from humans or animals, stem cells that can differentiate into different types of cells, or cells from various cell lines. Some types of live cells can have a limited lifespan, and some types can be propagated indefinitely. In some examples, the concentration of live cells in the bio-ink can be from about 1 million cells/mL to about 4 million cells/mL. In other examples, the concentration of live cells can be from about 1 million cells/mL to about 2 million cells/mL, or from about 2 million cells/mL to about 4 million cells/mL, or from about 3 million cells/mL to about 4 million cells/mL.

The polymer that is included in the bio-ink can have several effects, including increasing the effective surface tension of the bio-ink when under high shear stress, reducing settling and aggregation of cells in the bio-ink, cushioning cells to increase cell viability in bio-ink droplets printed on a substrate, reducing splattering and splashing of ink droplets, and others. In some cases, when a droplet of bio-ink is printed on a substrate, the bio-ink can form a central droplet on the substrate and multiple smaller “satellite” droplets around the central droplet. The satellite droplets can be caused by splashing of the bio-ink after impacting the tablet, or separation of the droplet into multiple smaller droplets in the air before impacting the tablet, or both. In some examples, a droplet of bio-ink can separate into smaller droplets while the droplet travels through the air before impacting the substrate, and the smaller droplets can become somewhat scattered in the air. Thus, some of the smaller droplets can impact the tablet outside the boundary of the main central droplet on the substrate. However, it has been found that adding the polymer to the bio-ink, as described herein, can reduce the number of these satellite droplets compared to bio-ink that does not include the polymer. In some examples, the polymer can reduce satellite droplets both due to the splashing of the bio-ink and separation and scattering of droplets in the air. Thus, the bio-ink described herein can provide more coherent and well-formed droplets printed on a substrate compared to bio-ink that does not include the polymer.

As explained above, the polymer can include a variety of types of polymer including polyvinylpyrrolidone, polyvinyl alcohol, polyvinyl sulfate, polyethylene glycol, polyester, poly(dimethylsiloxane), cellulose, polysaccharide, or a combination thereof. In some examples, the polymer can be one of these types of polymers, without any of the others. In other examples, the polymer can be a combination of two of these types of polymers. In a particular example, the polymer can be 90 wt % or more of one of the listed types of polymers, with 10 wt % or less of a second type of polymer or combination of multiple other polymers. In a certain example, the polymer can include polyvinylpyrrolidone. In another example, the polymer can be polyvinylpyrrolidone, and the bio-ink may not include any of the other listed types of polymer.

Depending on the type of polymer that is included in the bio-ink, various molecular weights of polymer can be useful. In some examples, the polymer can have a weight average molecular weight from about 100,000 Mw to about 500,000 Mw. In further examples, the molecular weight can be from about 200,000 Mw to about 400,000 Mw or from about 300,000 Mw to about 400,000. In some particular examples, the polymer can include polyvinylpyrrolidone having a weight average molecular weight from about 300,000 Mw to about 400,000 Mw, or from about 340,000 Mw to about 380,000 Mw, or about 360,000 Mw.

The amount of polymer that is included in the bio-ink can be sufficient to provide the effects described above, such as increased cell viability and reduced splashing. In some examples, including too much polymer in the bio-ink may interfere with the printability of the ink. For example, including too much polymer in the bio-ink can overly increase the viscosity of the bio-ink and make the bio-ink difficult to print in some examples. In certain examples, the amount of polymer in the bio-ink can be from about 1 wt % to about 3 wt % with respect to the total weight of the bio-ink. In further examples, the amount of polymer can be from about 1 wt % to about 2.5 wt %, or from about 1.5 wt % to about 2.5 wt %, or from about 2 wt % to about 2.5 wt % with respect to the total weight of the bio-ink.

The bio-ink can also include a buffer solution that is suitable for live cells. A variety of buffer formulations can be used. In some examples, the buffer solution can be made up of small molecules as described above. Some example buffer solutions include phosphate buffered saline (PBS), citric acid buffer, Tris buffer, HEPES buffer, CABS buffer, CAPS buffer, AMP buffer, CAPSO buffer, CHES buffer, AMPSO buffer, TABS buffer, AMPD buffer, TAPS buffer, HEPBS buffer, Bicine buffer, Gly-Gly buffer, Tricine buffer, EPPS buffer, TEA buffer, POPSO buffer, HEPPSO buffer, TAPSO buffer, MOBS buffer, DIPSO buffer, HEPES buffer, TES buffer, MOPS buffer, BES buffer, Bis-Tris Propane buffer, MOPSO buffer, PIPES buffer, ACES buffer, ADA buffer, Bis-Tris buffer, MES buffer, and combinations thereof. The concentration of the buffer solution can be an appropriate concentration for live cells.

The type of substrate that is used for printing can depend on the particular application for the printed bio-ink. In some examples, the substrate can include a well-plate, a petri dish, a layer of previously-printed cells, glass such as a glass slide, hydrogel, a cellular scaffold, or another type of substrate. In certain examples, the substrate can include an engineered tissue formed of previously-printed cells. In some cases, a bio-ink printer component may be useable with multiple different types of substrates. Additionally, in some examples the bio-ink ejector can have a relative clearance over the substrate compared to other types of printers such as paper inkjet printers. Specifically, in some examples the substrate can be at a distance from about 5 mm to about 20 mm away from the ejection nozzle of the bio-in ejector. In further examples, the substrate can be positioned at a distance from about 5 mm to about 10 mm, or from about 5 mm to about 15 mm, or from about 10 mm to about 20 mm, or from about 15 mm to about 20 mm, away from the ejection nozzle. In some examples, the ejection nozzle can be above the substrate and oriented to eject the bio-ink downward onto the substrate. However, in other examples, a different orientation can be used.

The bio-ink ejector can include an ejection nozzle and a thermal resistor. As explained above, the thermal resistor can heat the bio-ink to form a vapor bubble that forces bio-ink out of the ejection nozzle. Thus, the mechanical energy used to eject a droplet of bio-ink from the ejector can be provided by the vapor bubble formed by the thermal resistor. The thermal resistor can be made of metal, metal oxide, or another conductive material, in some examples. In certain examples, the thermal resistor can be formed by a thin-film deposition process. A stack of thin film layers can be formed that can include a thin layer of metal, metal oxide, or other conductive material to act as a thermal resistor, and additional layers can also be included such as an oxide layer, a passivation layer, electrically conductive traces, or combinations thereof. The thermal resistor can be activated by passing an electric current through the thermal resistor to generate heat.

In many examples, the bio-ink printer component can include multiple bio-ink ejectors that can be arranged in arrays or columns. Thus, the bio-ink can be ejected from multiple ejection nozzles to allow the bio-ink to be printed more quickly over a larger area of a substrate. The individual bio-ink ejectors can be separately controllable, so that individual thermal resistors can be activated at a desired time to allow fine control over the location where bio-ink is printed. In some examples, multiple bio-ink ejectors can be connected to a common bio-ink supply, such as a bio-ink supply slot that is fluidly connected to the individual ejection chambers of the individual bio-ink ejectors. Thus, a single type of bio-ink can be printed from many bio-ink ejectors. The combination of one supply of bio-ink and many bio-ink ejectors can make up a single print head of a bio-ink printer. In further examples, the bio-ink printer can include multiple print heads that may print multiple different types of bio-ink, or the same type of bio-ink.

The size of elements of the bio-ink ejector are not particularly limited. However, as mentioned above, in some examples the ejection nozzle can have a diameter from about 20 μm to about 80 μm. In further examples, the ejection nozzle can have a diameter from about 20 μm to about 50 μm or from about 50 μm to about 80 μm. The thermal resistor can have a width or diameter from about 20 μm to about 200 μm in some examples, or from about 50 μm to about 200 μm or from about 100 μm to about 200 μm, or from about 50 μm to about 100 μm, in other examples. The ejection chamber can have an internal width, length, or height (i.e., any internal dimension) from about 20 μm to about 400 μm in some examples, or from about 20 μm to about 300 μm, or from about 50 μm to about 300 μm, or from about 100 μm to about 300 μm, or from about 200 μm to about 400 μm, in other examples.

Bio-ink printers can also include a variety of other components not illustrated in the figures herein. Additional components may include a print head assembly, a moveable print head carriage, a substrate transport assembly, an electronic controller to control the activation of bio-ink ejectors and the motion of print heads and/or substrates, a power supply, communication or networking modules, and others. In some examples, an electronic controller can include a processor, memory components including volatile and/or non-volatile memory components, electronics for communicating with and controlling bio-ink ejectors, print head assemblies, carriages, substrate transport assemblies, and so on. In certain examples, the electronic controller can receive data from a host system, such as a computer, and temporarily store data in memory. The data can include a representation of locations at which bio-ink is to be printed. The electronic controller can control the bio-ink ejectors to print a pattern of droplets of bio-ink on the substrate based on the data.

### Methods of Printing Bio-Ink

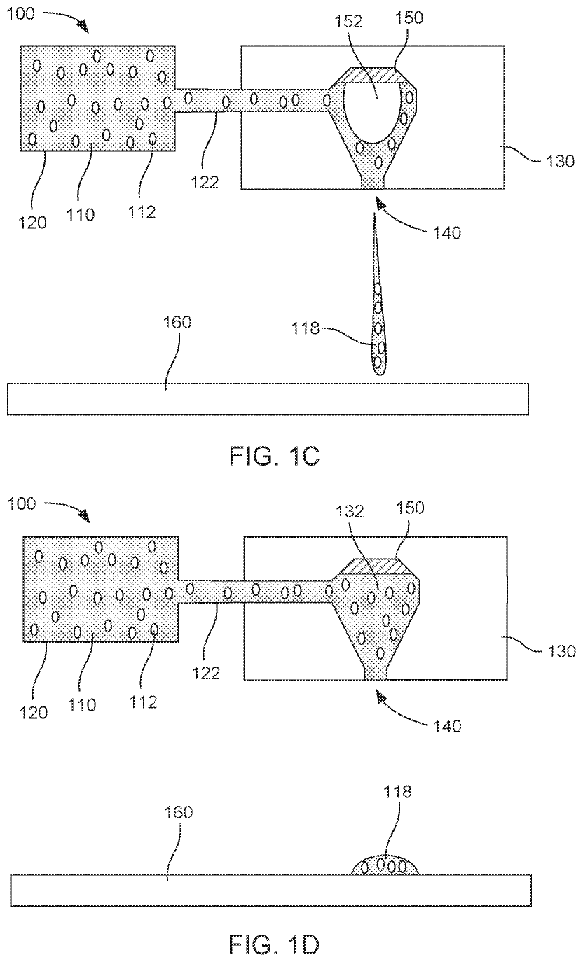

The bio-inks described herein can be used in methods for printing bio-ink. In some examples, the methods can use a bio-ink printer component such as the bio-ink printer components described above. FIG. 2 is a flowchart illustrating one example method 200 of printing bio-ink. This method can include ejecting a droplet of a bio-ink from a bio-ink ejector including an ejection nozzle and a thermal resistor positioned to heat the bio-ink to form a vapor bubble to eject the droplet of bio-ink from the ejection nozzle, wherein the bio-ink includes live cells, a buffer solution that is suitable for live cells, and a polymer including polyvinylpyrrolidone, polyvinyl alcohol, polyvinyl sulfate, polyethylene glycol, polyester, poly(dimethylsiloxane), cellulose, polysaccharide, or a combination thereof 210.

In various examples, the methods of printing bio-ink can include bio-inks having any of the ingredients and properties described above. Additionally, the methods can be performed using bio-ink ejectors and bio-ink printers that include any of the components and features described above.

As mentioned above, in some examples the bio-ink can stretch to form a relatively long filament extending from the ejection nozzle of the bio-ink ejector. In some examples, the filament can have a length from about 2 mm to about 6 mm before detaching from the ejection nozzle. In further examples, the filament can be from about 3 mm to about 6 mm, or from about 4 mm to about 6 mm, or from about 2 mm to about 5 mm, or from about 3 mm to about 5 mm, before detaching from the ejection nozzle. This filament length can depend, in part, on the viscosity and surface tension of the bio-ink. In some examples, the bio-ink can have a viscosity from about 0.1 centipoise to about 15 centipoise, or from about 0.5 centipoise to about 10 centipoise, or from about 1 centipoise to about 8 centipoise, or from about 3 centipoise to about 8 centipoise. The surface tension of the bio-ink can be from about 20 dyn/cm to about 60 dyn/cm, or from about 30 dyn/cm to about 50 dyn/cm, or from about 35 dyn/cm to about 50 dyn/cm, in some examples.

In some examples, the drop volume of bio-ink drops printed from the bio-ink ejectors can be controlled by varying the amount of electric current supplied to the thermal resistor. A variety of drop volumes can be used, such as from about 1 nL to about 100 nL, or from about 5 nL to about 100 nL, or from about 20 nl to about 80 nL, or from about 20 nL to about 60 nL. In further examples, the drop velocity can also be controlled. In some examples, drops of bio-ink can be printed with a drop velocity from about 10 m/s to about 40 m/s, or from about 20 m/s to about 40 m/s, or from about 20 m/s to about 30 m/s.

The size of the bio-ink drop on the substrate, after being printed, can depend on the drop volume. Additionally, the number of satellite droplets that form can also affect the size of the main bio-ink drop (since the satellite droplets reduce the amount of bio-ink in the main drop). In various examples, a drop of bio-ink printed on a substrate can form a main drop that has a diameter from about 100 μm to about 2 mm, or from about 200 μm to about 1.5 mm, or from about 400 μm to about 1.5 mm, or from about 500 μm to about 1.5 mm. In some particular examples, these drops can be printed onto a glass substrate.

Satellite droplets can be much smaller droplets that either separate from the main drop while the drop is travelling toward the substrate, or small droplets that form from splashing of the bio-ink after impacting the substrate. In some examples, satellite droplets, if any form, can make up a relatively small portion of the total surface area of the substrate covered by the bio-ink. In certain examples, satellite droplets can cover from 0% to about 40% of the total substrate surface area that is covered by a drop of bio-ink printed from a bio-ink ejector. In further examples, satellite droplets can cover from 0% to about 30%, or from 0% to about 20%, or from 0% to about 10%, or from 0% to about 5%, of the total substrate surface area that is covered by the drop of bio-ink. The remaining surface area can be covered by the main central drop of bio-ink. In some examples, the addition of the polymer in the bio-ink can help to reduce the number of satellite droplets. This reduction of satellite droplets can also be helpful to prevent cross-contamination between bio-ink droplets printed one near another.

When the bio-ink is printed onto a substrate, the cell viability of cells in the printed bio-ink drop can be high. Cell viability can be defined as the portion of cells that are alive in a volume of bio-ink that was printed on a substrate out of the total cells that were alive in that volume of ink before being printed. If all of the cells survive being printed onto the substrate, then the cell viability is 100%. In some examples, the cell viability of cells in a printed drop of the bio-ink can be from about 90% to about 100%. In further examples, the cell viability can be from about 90% to about 95%, or from about 95% to about 100%.

### Multi-Fluid Live Cell Printing Systems

Multi-fluid live cell printing systems can include multiple types of bio-ink and bio-ink ejectors to print these multiple bio-inks onto a substrate. One or multiple types of the bio-inks used in the systems can be a bio-ink as described herein. In some examples, the systems can include two or more different bio-inks, and the bio-inks can include different types of live cells. Such systems can be used to selectively print multiple different types of living cells in desired locations on a substrate. In some examples, a multi-fluid live cell printing system can be a group of multiple print cartridges, where the print cartridges include different bio-inks including living cells. In other examples, the multi-fluid live cell printing system can be bio-ink printer that includes multiple print cartridges

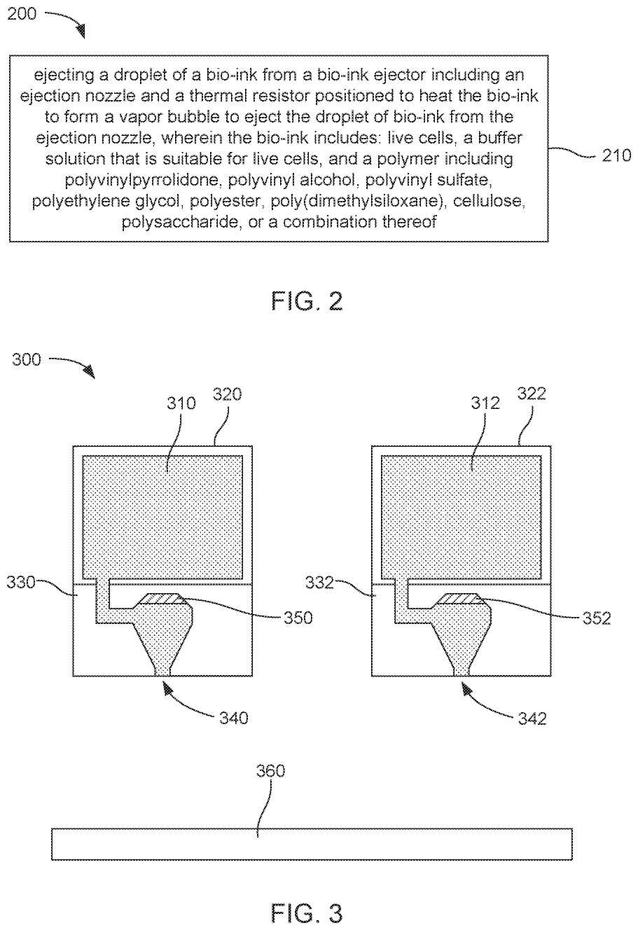

FIG. 3 shows a schematic cross-sectional view of an example multi-fluid live cell printing system 300. This system includes a first bio-ink 310 in a first bio-ink reservoir 320. The first bio-ink includes a first type of live cells, a buffer solution that is suitable for live cells, and a polymer that includes polyvinylpyrrolidone, polyvinyl alcohol, polyvinyl sulfate, polyethylene glycol, polyester, poly(dimethylsiloxane), cellulose, polysaccharide, or a combination thereof. The system also includes a second bio-ink 312 in a second bio-ink reservoir 322. The second bio-ink includes a buffer solution that is also suitable for live cells. A first bio-ink ejector 330 is connected to the first bio-ink reservoir so that the first bio-ink ejector can eject the first bio-ink from the first bio-ink reservoir. The first bio-ink ejector includes a first ejection nozzle 340 and a first thermal resistor 350 positioned to heat the first bio-ink to form a vapor bubble to eject a droplet of the first bio-ink from the first ejection nozzle. Similarly, a second bio-ink ejector 332 is connected to the second bio-ink reservoir to eject the second bio-ink. In this example, the second bio-ink ejector includes a second ejection nozzle 342 and a second thermal resistor 352. In some examples, the second bio-ink can include a second type of live cells and a polymer. The polymer can be a different polymer from the polymer in the first bio-ink, or the same polymer. This system also includes a substrate 360 positioned to receive bio-ink droplets printed from the bio-ink ejectors.

In certain examples, the multi-fluid live cell printing system can include a cassette that includes the first and second bio-ink reservoirs and the first and second bio-ink ejectors. The cassette may be packaged with a first and second bio-ink in the reservoirs. Alternatively, the cassette can be provided empty and a user can introduce a first and second bio-ink into the reservoirs. The cassette can then be loaded in a dispenser that can include other components such as a power supply, electronic controller, a carriage, a substrate transport assembly, and so on. The cassette can be a disposable cassette, designed for one-time use, so that subsequent bio-inks are not contaminated by cells from previously used bio-inks. In further examples, the cassette can include four bio-ink reservoirs and four bio-ink ejectors. In still further examples, the cassette can include eight bio-ink reservoirs and eight bio-ink ejectors.

It is noted that, as used in this specification and the appended claims, the singular forms “a,” “an,” and “the” include plural referents unless the content clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and can be determined based on experience and the associated description herein.

As used herein, a plurality of items, structural elements, compositional elements, and/or materials may be presented in a common list for convenience. However, these lists should be construed as though the individual members of the list are individually identified as separate and unique members. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include the numerical values explicitly recited as the limits of the range, and also to include all the individual numerical values or sub-ranges encompassed within that range as if the numerical values and sub-ranges are explicitly recited. For example, a weight ratio range of 1 wt % to 20 wt % should be interpreted to include the explicitly recited limits of 1 wt % and 20 wt %, and also to include individual weights such as 2 wt %, 11 wt %, 14 wt %, and sub-ranges such as 10 wt % to 20 wt %, 5 wt % to 15 wt %, etc.

### EXAMPLES

The following examples illustrate the technology of the present disclosure. However, it is to be understood that the following is merely illustrative of the application of the principles of the presented formulations and methods. Numerous modifications and alternative methods may be devised without departing from the scope of the present disclosure. The appended claims are intended to cover such modifications and arrangements. Thus, while the technology has been described above with particularity, the following examples provide further detail in connection with what are presently deemed to be certain acceptable examples.

### Example 1—Influence of Cell Concentration and Droplet Volume on Printing

A series of 5 control bio-inks were prepared. The first bio-ink was a pure 1× solution of phosphate buffered saline (PBS) without any cells added. The other bio-inks were made up of a 1× solution of PBS with live cells added at a concentration of 1 million cells/mL, 2 million cells/mL, 3 million cells/mL, and 4 million cells/mL, respectively. These bio-inks were then loaded in an HP D300e Digital Dispenser and droplets of the bio-inks were printed onto tissue-cultured well plates. All of the bio-inks were printed with three different drop volumes, including 20 nL, 40 nL, and 60 nL. After the droplets were printed on the well plates, the droplets were observed using a microscope.

The elongation of the filament formed when printing the droplets was measured. It was found that increasing cell concentration resulted in shorter filament elongation. The ink with 0 cells/mL had a filament length of 3.72 mm. The ink with 1 million cells/mL had a filament length of 3.39 mm. The ink with 2 million cells/mL had a filament length of 2.48 mm. The ink with 3 million cells/mL had a filament length of 2.33 mm.

### Example 2—Printing of Bio-Ink Including Polyvinylpyrrolidone Polymer

Two sample bio-inks were prepared that included polyvinylpyrrolidone, which is an example of the polymer in the bio-inks described herein. The sample bio-inks were made up of a 1× solution of PBS with either 1 wt % or 2 wt % of polyvinylpyrrolidone added. These sample bio-inks were printed from the HP D300e Digital Dispenser. A high speed camera was used to photograph the droplets in the process of being ejected from the dispenser and while impacting the substrate. For comparison, the control bio-ink made up of pure 1×PBS solution was also printed and photographed.

High speed photographs of the droplets being ejected from the dispenser showed that the bio-inks formed a filament of fluid attached to the ejector nozzle which stretched for a certain distance before the filament detached and the droplet continued to travel as a free droplet through the air. The length of the filament formed by the control bio-ink (1×PBS, 0 wt % PVP) was about 0.2 mm. The length of the filament formed by the sample bio-ink having 1 wt % PVP was about 1.6 mm. The length of the filament formed by the sample bio-ink having 2 wt % PVP was about 4.4 mm. Thus, the increasing concentration of PVP up to 2 wt % appears to increase the length of the filament formed by the bio-ink before the filament detaches from the ejection nozzle. The length of time from firing the ejector until the filament detached was about 80 us for the bio-ink having 0 wt % PVP, about 150 us for the bio-ink having 1 wt % PVP, and about 250 us for the bio-ink having 2 wt % PVP. Increasing the concentration of PVP in the bio-ink also appears to slow down the overall velocity of the bio-ink droplets.

High speed photographs were also taken of the droplets impacting the substrate. These photographs showed that the control bio-ink having 0 wt % PVP formed a relatively large splash when the droplet impacted the substrate. The splash separated into many small satellite droplets which were temporarily suspended in the air and then fell to the substrate, with some satellite droplets falling outside the diameter of the main droplet. The sample bio-ink having 1 wt % PVP formed a relatively smaller splash. The splash partially separated to form some smaller droplets, but the smaller droplets stayed more consolidated with the main droplet compared to the control bio-ink. The sample bio-ink having 2 wt % PVP produced an even smaller splash, which had very little separation into smaller droplets, and mostly remained consolidated as a single main droplet.

High speed photographs were also taken that included the droplets impacting the substrate and the vertical space between the substrate and the ejector. When the bio-ink droplets were ejected, some smaller satellite droplets separated away from the main droplet while the droplets were travelling through the air toward the substrate. Some small satellite droplets were still travelling through the air at the moment when the main droplet impacted the substrate. These photographs show the relative amounts of lateral spread of the satellite droplets in the air. When the control bio-ink having 0 wt % PVP was printed, a large number of satellite droplets formed in the air, and the satellite droplets spread out laterally outside the boundaries of the location where the main droplet impacted the substrate. The sample bio-ink having 1 wt % PVP also produced a significant amount of satellite droplets, but the satellite droplets were not spread as far to the sides as with the control bio-ink. The sample bio-ink having 2 wt % PVP appears to have produced less satellite droplets overall, and the satellite droplets that were produced did not spread as far as with the 0 wt % PVP or 1 wt % PVP bio-inks. Thus, increasing the concentration of PVP up to 2 wt % appears to increase the precision of printing by reducing the spread of satellite droplets. Without being bound to a particular mechanism, this may be due to the longer filament formed by the bio-ink having 2 wt % PVP. The filament keeps the bio-ink consolidated until the filament breaks, and after the filament breaks there is a shorter time for satellite droplets to form and spread out before impacting the substrate.

### Example 3—Influence of Polymer on Droplet Shape and Resolution

The same three bio-inks (include the control bio-ink having 0 wt % PVP, the sample bio-ink having 1 wt % PVP, and the sample bio-ink having 2 wt % PVP) were printed using the HP D300e digital dispenser and the printed droplets were observed with a microscope. The control bio-ink having 0 wt % PVP formed a relatively small main droplet with many satellite droplets spread around the main droplet on the substrate. The bio-ink was printed at three different droplet volumes, including 20 nL, 40 nL, and 60 nL. The 20 nL droplet formed a main droplet having a diameter of about 594 μm; the 40 nL droplet formed a main droplet having a diameter of about 772 μm; and the 60 nL droplet formed a main droplet having a diameter of about 921 μm. All of the main droplets were surrounded by many satellite droplets.

The sample bio-ink having 1 wt % PVP was also printed at droplet volumes of 20 nL, 40 nL, and 60 nL. This sample bio-ink clearly formed far fewer satellite droplets, and the satellite droplets that formed were smaller than the satellite droplets formed by the control bio-ink. However, the main droplets printed with the 1 wt % PVP bio-ink were irregularly shaped and the diameter was difficult to measure.

The sample bio-ink having 2 wt % PVP was also printed with the same droplet volumes of 20 nL, 40 nL, and 60 nL. This sample bio-ink formed more regular-shaped circular main droplets, with a small number of satellite droplets that were also smaller in size compared to the satellite droplets of the control bio-ink. The 20 nL droplet formed a main droplet having a diameter of about 713 μm; the 40 nL droplet formed a main droplet having a diameter of about 911 μm; and the 60 nL droplet formed a main droplet having a diameter of about 1110 μm. These main droplet diameters were comparatively larger than the main droplet diameters of the sample ink, indicating that a smaller portion of the droplet volume was converted to satellite droplets and most of the droplet volume remained consolidated in the main printed droplet.

### Example 4—Influence of Cells on Droplet Shape and Resolution

A series of four sample bio-inks were prepared including 2 wt % PVP in a 1×PBS solution and four different concentrations of live cells. The cell concentrations used were 0 cells/mL, 1 million cells/mL, 2 million cells/mL, and 3 million cells/mL. These sample bio-inks were printed using the HP D300e Digital Dispenser and the printed droplets were observed through a microscope. The concentration of cells appeared to have less impact on the droplet shape and resolution than the PVP concentration. The concentration of cells also had a larger impact on the filament elongation length than on the droplet shape and resolution.

### Example 5—Cell Proliferation

To test the proliferation of cells printed using the bio-inks described herein, the sample bio-inks having 2 wt % PVP and live cell concentrations of 1 million cells/mL, 2 million cells/mL, and 3 million cells/mL, respectively, were printed on tissue-culture well plates. The printed bio-ink was then left for 3 days to allow the cells to proliferate. As a control, solutions of cells in 1×PBS were prepared and placed onto tissue-cultured well plates using a pipette (i.e., not printed). A solution of 1 million cells/mL in 1×PBS was placed on a tissue-culture well plate to be used as a control for the printed bio-ink having 2 wt % PVP and 1 million cells/mL. Similarly, control solutions having 2 million cells/mL and 3 million cells/mL were also placed on tissue-culture well plates. Thus, the well plates having printed cells can be directly compared to the control well plates having non-printed cells. After three days, the printed cells appeared to have proliferated at a comparable rate to the non-printed cells. This indicates that the process of printing the cells in a bio-ink that includes 2 wt % PVP does not impact the proliferation ability of the cells.

While the present technology has been described with reference to certain examples, various modifications, changes, omissions, and substitutions can be made without departing from the disclosure. It is intended, therefore, that the disclosure be limited by the scope of the following claims.

What is claimed is:

1. A bio-ink printer component comprising:
a bio-ink comprising:
a buffer solution that is suitable for live cells, and
a polymer comprising polyvinylpyrrolidone, polyvinyl alcohol, polyvinyl sulfate, polyethylene glycol, polyester, poly(dimethylsiloxane), cellulose, polysaccharide, or a combination thereof; and
a bio-ink ejector fluidly connected or connectable to the bio-ink, wherein the bio-ink ejector comprises an ejection nozzle and a thermal resistor positioned to heat the bio-ink to form a vapor bubble to eject a droplet of bio-ink from the ejection nozzle.

2. The bio-ink printer component of claim 1, wherein the bio-ink further comprises live cells.

3. The bio-ink printer component of claim 1, wherein the polymer is present in an amount from about 1 wt % to about 3 wt % with respect to the total weight of the bio-ink.

4. The bio-ink printer component of claim 1, wherein the buffer solution comprises phosphate buffered saline (PBS), citric acid buffer, Tris buffer, HEPES buffer, CABS buffer, CAPS buffer, AMP buffer, CAPSO buffer, CHES buffer, AMPSO buffer, TABS buffer, AMPD buffer, TAPS buffer, HEPBS buffer, Bicine buffer, Gly-Gly buffer, Tricine buffer, EPPS buffer, TEA buffer, POPSO buffer, HEPPSO buffer, TAPSO buffer, MOBS buffer, DIPSO buffer, HEPES buffer, TES buffer, MOPS buffer, BES buffer, Bis-Tris Propane buffer, MOPSO buffer, PIPES buffer, ACES buffer, ADA buffer, Bis-Tris buffer, MES buffer, or a combination thereof.

5. The bio-ink printer component of claim 1, further comprising a substrate positioned to receive the droplet of the bio-ink ejected from the ejection nozzle, wherein the substrate comprises a well-plate, a petri dish, a layer of previously-printed cells, glass, hydrogel, or a cellular scaffold, and wherein the substrate is positioned or positionable at a distance from about 5 mm to about 20 mm away from the ejection nozzle.

6. The bio-ink printer component of claim 5, wherein the substrate is positioned at a distance from about 5 mm to about 20 mm away from the ejection nozzle.

7. The bio-ink printer component of claim 1, wherein the ejection nozzle has a width from about 20 micrometers to about 80 micrometers.

8. A method of printing bio-ink comprising ejecting a droplet of a bio-ink from a bio-ink ejector comprising an ejection nozzle and a thermal resistor positioned to heat the bio-ink to form a vapor bubble to eject the droplet of bio-ink from the ejection nozzle, wherein the bio-ink comprises:
live cells,
a buffer solution that is suitable for live cells, and
a polymer comprising polyvinylpyrrolidone, polyvinyl alcohol, polyvinyl sulfate, polyethylene glycol, polyester, poly(dimethylsiloxane), cellulose, polysaccharide, or a combination thereof.

9. The method of claim 8, wherein the live cells are present in the bio-ink at a concentration from about 1 million cells/mL to about 4 million cells/mL, and wherein the polymer is present in an amount from about 1 wt % to about 3 wt % with respect to the total weight of the bio-ink.

10. The method of claim 8, wherein the buffer solution comprises phosphate buffered saline (PBS), citric acid buffer, Tris buffer, HEPES buffer, CABS buffer, CAPS buffer, AMP buffer, CAPSO buffer, CHES buffer, AMPSO buffer, TABS buffer, AMPD buffer, TAPS buffer, HEPBS buffer, Bicine buffer, Gly-Gly buffer, Tricine buffer, EPPS buffer, TEA buffer, POPSO buffer, HEPPSO buffer, TAPSO buffer, MOBS buffer, DIPSO buffer, HEPES buffer, TES buffer, MOPS buffer, BES buffer, Bis-Tris Propane buffer, MOPSO buffer, PIPES buffer, ACES buffer, ADA buffer, Bis-Tris buffer, MES buffer, or a combination thereof.

11. The method of claim 8, wherein the droplet forms a filament stretching to a distance from about 2 mm to about 6 mm from the ejection nozzle before the filament separates from the ejection nozzle.

12. The method of claim 8, wherein the droplet is ejected onto a substrate and wherein the live cells have a viability from about 90% to about 100% after being ejected onto the substrate.

13. A multi-fluid live cell printing system comprising:
a first bio-ink reservoir containing a first bio-ink, wherein the first bio-ink comprises:
a first type of live cells,
a buffer solution that is suitable for live cells, and
a polymer comprising polyvinylpyrrolidone, polyvinyl alcohol, polyvinyl sulfate, polyethylene glycol, polyester, poly(dimethylsiloxane), cellulose, polysaccharide, or a combination thereof;
a second bio-ink reservoir containing a second bio-ink, wherein the second bio-ink comprises a buffer solution that is also suitable for live cells;
a first bio-ink ejector to eject the first bio-ink from the first bio-ink reservoir, wherein the first bio-ink ejector comprises a first ejection nozzle and a first thermal resistor positioned to heat the first bio-ink to form a vapor bubble to eject a droplet of the first bio-ink from the first ejection nozzle; and
a second bio-ink ejector to eject the second bio-ink from the second bio-ink reservoir.

14. The multi-fluid live cell printing system of claim 13, wherein the polymer is present in the first bio-ink at an amount from about 1 wt % to about 3 wt % with respect to the total weight of the first bio-ink.

15. The multi-fluid live cell printing system of claim 13, wherein the second bio-ink includes a second type of live cells, a polymer including polyvinylpyrrolidone, polyvinyl alcohol, polyvinyl sulfate, polyethylene glycol, polyester, poly(dimethylsiloxane), cellulose, polysaccharide, or a combination thereof, and wherein the second bio-ink ejector comprises a second ejection nozzle and a second thermal resistor positioned to heat the second bio-ink to form a vapor bubble to eject a droplet of the second bio-ink from the second ejection nozzle.
